(19) United States
(12) Patent Application Publication
(10) Pub. No.: US 2015/0342788 A1
(43) Pub. Date: Dec. 3, 2015

(54) Bandage Assembly

(71) Applicants: Mitchell Watz, Festus, MO (US); Don Dinges, Festus, MO (US)

(72) Inventors: Mitchell Watz, Festus, MO (US); Don Dinges, Festus, MO (US)

(21) Appl. No.: 14/293,788

(22) Filed: Jun. 2, 2014

(51) Int. Cl. A61F 13/02

(52) U.S. Cl. CPC A61F 13/0243; A61F 13/025; A61F 2013/00217

(57) ABSTRACT

A bandage assembly for covering a wound without sticking to the wound includes a strip that may be positioned over a wound. The strip has a concavely arcuate area with respect to a bottom surface of the strip. The concavely arcuate area of the strip does not contact the wound. An adhesive layer is coupled to the bottom surface of the strip. The adhesive layer engages a user's skin. The strip is retained on the user's skin.

### BACKGROUND OF THE DISCLOSURE

### Field of the Disclosure

The disclosure relates to bandage devices and more particularly pertains to a new bandage device for covering a wound without sticking to the wound.

### SUMMARY OF THE DISCLOSURE

An embodiment of the disclosure meets the needs presented above by generally comprising a strip that may be positioned over a wound. The strip has a concavely arcuate area with respect to a bottom surface of the strip. The concavely arcuate area of the strip does not contact the wound. An adhesive layer is coupled to the bottom surface of the strip. The adhesive layer engages a user's skin. The strip is retained on the user's skin.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a bandage assembly according to an embodiment of the disclosure.

FIG. 2 is a top view of an embodiment of the disclosure.

FIG. 3 is a bottom view of an embodiment of the disclosure.

FIG. 4 is a right side view of an embodiment of the disclosure.

FIG. 5 is a top view of an alternative embodiment of the disclosure.

FIG. 6 is a cross sectional view taken along line 6-6 of FIG. 5 of an embodiment of the disclosure.

### DESCRIPTION OF THE PREFERRED EMBODIMENT

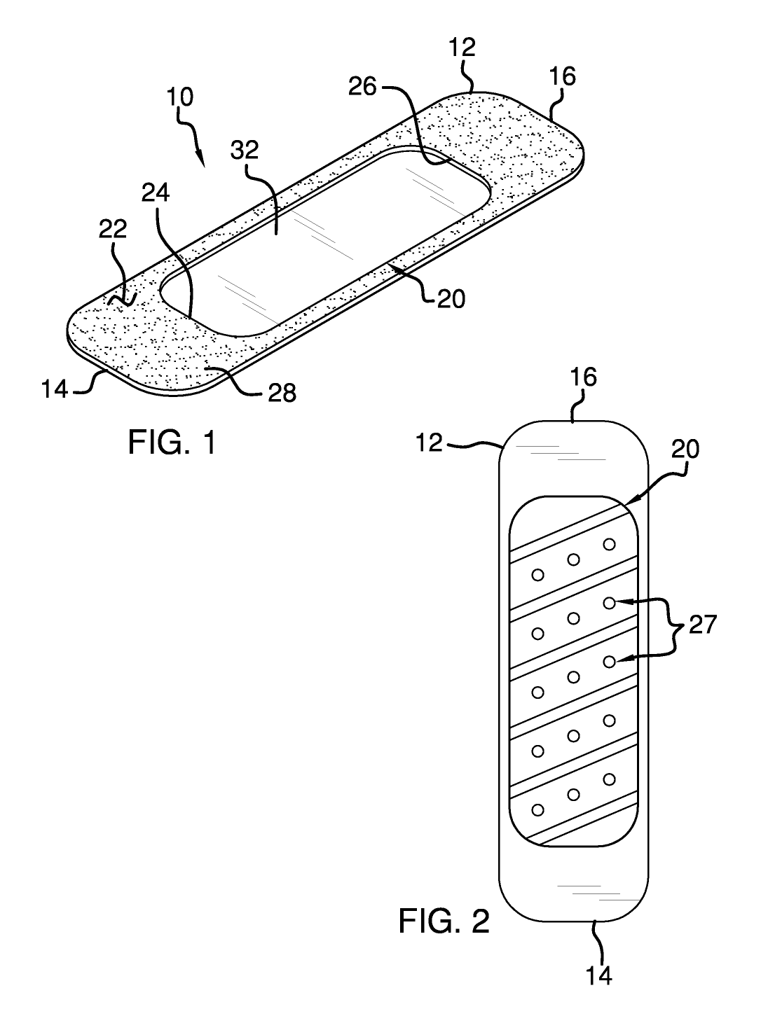

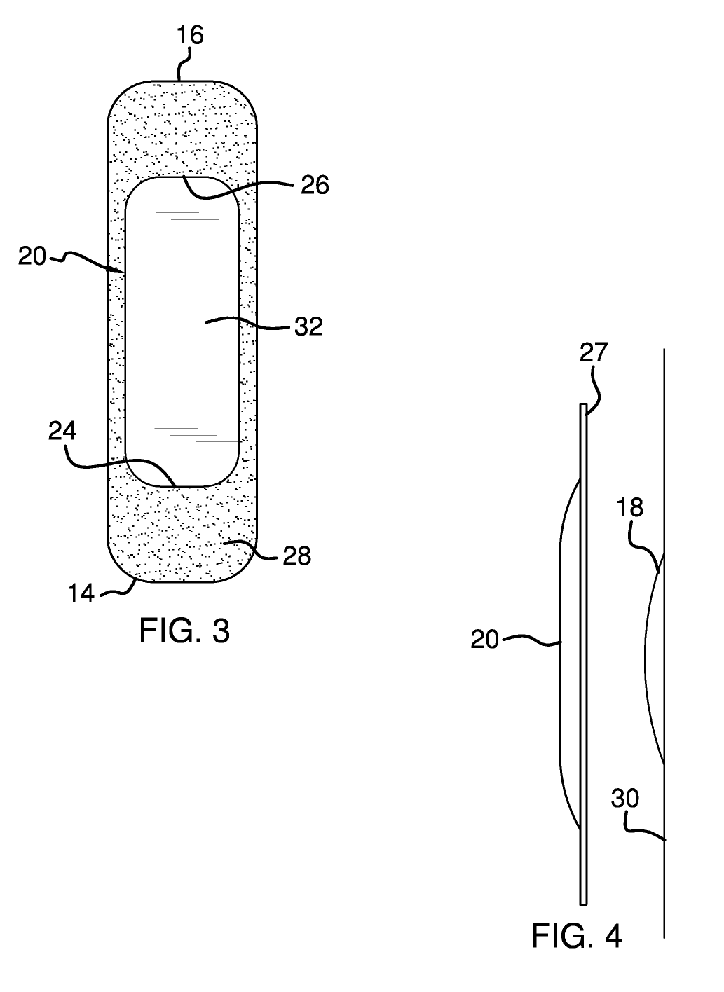

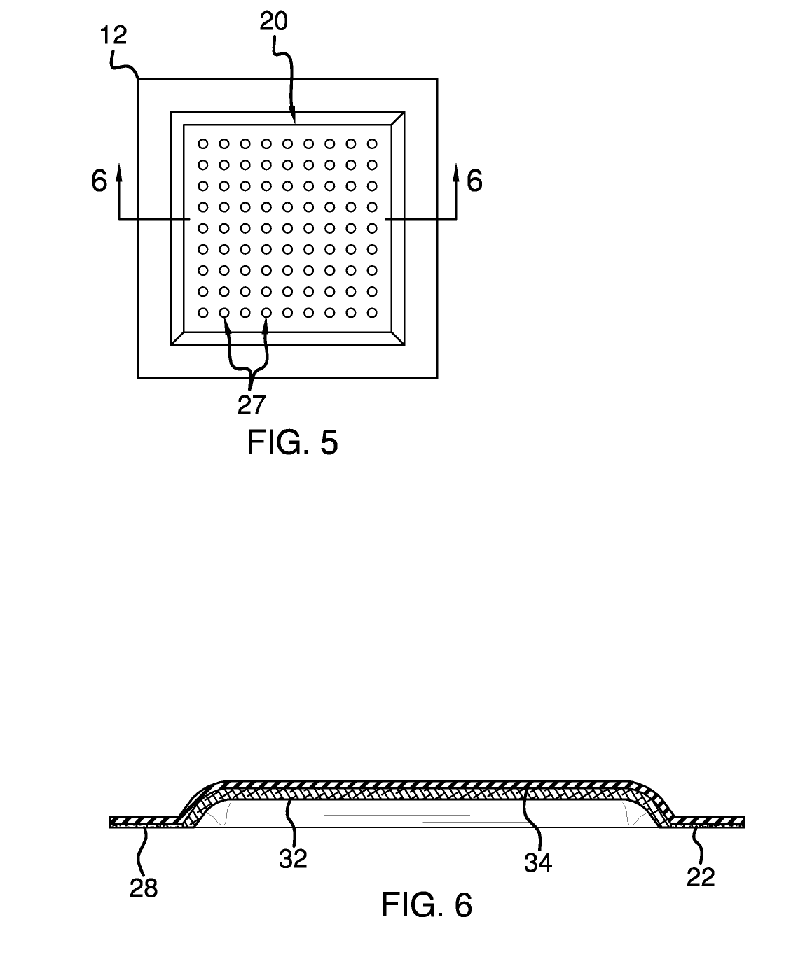

With reference now to the drawings, and in particular to FIGS. 1 through 6 thereof, a new bandage device embodying the principles and concepts of an embodiment of the disclosure and generally designated by the reference numeral 10 will be described.

As best illustrated in FIGS. 1 through 6, the bandage assembly 10 generally comprises a strip 12 that has a first end 14 and a second end 16. The strip 12 is elongated. Additionally, the strip 12 may be positioned over a wound 18. The strip 12 has a concavely arcuate area 20 with respect to a bottom surface 22 of the strip 12. The concavely arcuate area 20 of the strip 12 does not contact the wound 18. Alternatively, the strip 12 may have a length and a width that are similar. The strip 12 may have a square shape.

The concavely arcuate area 20 of the strip 12 has a primary end 24 and a secondary end 26. The concavely arcuate area 20 of the strip 12 is elongated. Moreover, the concavely arcuate area 20 of the strip 12 is centrally positioned on the strip 12. Each of the primary 24 and secondary 26 ends of the concavely arcuate area 20 of the strip 12 are positioned proximate an associated one of the first 14 and second 16 ends of the strip 12.

The concavely arcuate area 20 of the strip 12 is perforated. The perforations 27 allow air to contact the wound 18 when the strip 12 is positioned over the wound 18. The concavely arcuate area 20 of the strip 12 may be comprised of a rigid material. Moreover, the concavely arcuate area 20 of strip 12 resists being compressed downwardly against the wound 18.

An adhesive layer 28 is coupled to the bottom surface 22 of the strip 12. The adhesive layer 28 completely covers the bottom surface 22 of the strip 12. Additionally, the adhesive layer 28 is discrete from the concavely arcuate area 20 of the strip 12. The adhesive layer 28 engages a user's skin 30 so the strip 12 is retained on the user's skin 30. The adhesive layer 28 may comprise a non-residual, medical grade adhesive of any conventional design.

A panel 32 is coupled to a lower surface 34 of the concavely arcuate area 20 of the strip 12. The panel 32 protects the wound 18 when the strip 12 is positioned over the wound 18. The panel 32 may be comprised of an absorbent material such as gauze or other similar material.

In use, the strip 12 is positioned over the wound 18 so the adhesive layer 28 engages the user's skin 30. The strip 12 remains over the wound 18 until the wound 18 has healed. The strip 12 is removable from the user's skin 30 without disturbing the wound 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A bandage assembly configured to cover a wound without sticking to the wound, said assembly comprising:
a strip configured to be positioned over the wound;
said strip having a concavely arcuate area with respect to a bottom surface of said strip such that said concavely arcuate area of said strip does not contact the wound; and
an adhesive layer coupled to said bottom surface of said strip, said adhesive layer engaging a user's skin such that said strip is retained on the user's skin.

2. The assembly according to claim 1, further comprising said strip having a first end and a second end, said strip being elongated.

3. The assembly according to claim 1, further comprising said concavely arcuate area of said strip having a primary end and a secondary end, said concavely arcuate area of said strip being elongated.

4. The assembly according to claim 1, further comprising said concavely arcuate area of said strip being centrally positioned on said strip.

5. The assembly according to claim 4, further comprising such that each of a primary and a secondary end of said concavely arcuate area is positioned proximate an associated one of a first and a second end of said strip.

6. The assembly according to claim 1, further comprising said adhesive layer completely covering said bottom surface of said strip.

7. The assembly according to claim 1, further comprising said adhesive layer being discrete from said concavely arcuate area of said strip.

8. The assembly according to claim 1, further comprising a panel coupled to a lower surface of said concavely arcuate area of said strip.

9. The assembly according to claim 8, further comprising said panel completely covering said lower surface of said concavely arcuate area of said strip.

10. The assembly according to claim 8, further comprising said panel protecting the wound when said strip is positioned over the wound.

11. A bandage assembly configured to cover a wound without sticking to the wound, said assembly comprising:
a strip having a first end and a second end, said strip being elongated, said strip being configured to be positioned over the wound;
said strip having a concavely arcuate area with respect to a bottom surface of said strip such that said concavely arcuate area of said strip does not contact the wound;
said concavely arcuate area of said strip having a primary end and a secondary end, said concavely arcuate area of said strip being elongated;
said concavely arcuate area of said strip being centrally positioned on said strip such that each of said primary and secondary ends of said concavely arcuate area is positioned proximate an associated one of said first and second ends of said strip;
an adhesive layer coupled to said bottom surface of said strip, said adhesive layer completely covering said bottom surface of said strip, said adhesive layer being discrete from said concavely arcuate area of said strip, said adhesive layer engaging a user's skin such that said strip is retained on the user's skin; and
a panel coupled to a lower surface of said concavely arcuate area of said strip, said panel protecting the wound when said strip is positioned over the wound.
